(19) United States
(12) Patent Application Publication
(10) Pub. No.: US 2018/0015019 A1
(43) Pub. Date: Jan. 18, 2018

(54) PROCESS FOR MAKING ORAL CARE COMPOSITIONS

(71) Applicant: The Procter & Gamble Company, Cincinnati, OH (US)

(72) Inventors: Swapna BASA, Beijing (CN); Hongmei YANG, Beijing (CN); Jianwei CUI, Beijing (CN); James Albert BERTA, West Chester, OH (US); Ross STRAND, Singapore (SG)

(21) Appl. No.: 15/649,861

(22) Filed: Jul. 14, 2017

(30) Foreign Application Priority Data
Jul. 15, 2016 (CN) PCT/CN2016/090087

(51) Int. Cl. A61K 8/73; A61Q 11/00; A61K 8/04; A61K 8/24

(52) U.S. Cl. CPC A61K 8/73; A61K 8/24; A61Q 11/00; A61K 8/042; A61K 2800/596; A61K 2800/48

(57) ABSTRACT

A process for making an oral care composition comprising the step of hydrating a linear sulfated polysaccharide in water in the absence of any salts and followed by adding inorganic salts therein.

### FIELD OF THE INVENTION

The present invention relates to an improved process for making oral care compositions.

### BACKGROUND OF THE INVENTION

Linear sulfated polysaccharides, such as for example carrageenan, have been reported in oral care composition as binding or thickening agents. Generally, the purpose of these agents is to thicken compositions to a desirable consistency and/or to prevent separation of the solid and liquid components, especially during the storage phase of the compositions. The thickening property is achieved upon the hydration of the thickening agent. The degree of hydration of the thickening agent can be measured by the viscosity of the resultant compositions.

Inorganic salts, such as for example, trisodium phosphate, monosodium phosphate, sodium monofluoride phosphate and tetra sodium pyrophosphate, can be used in oral care composition to provide desired benefits such as pH buffering and/or dental carries protection.

A traditional process for making oral care composition is by combining thickening agents and inorganic salts together into a vessel and mixing the resultant mixture while heating to an elevated temperature (e.g. 60° C.) until a homogeneous dispersion is formed. The residence time period for this mixing process is largely determined by the length of time needed for hydration.

Heating is a required step in the process in order to increase the hydration rate and/or help fully complete the hydration. However, one notable drawback to this existing process is that it takes time to cool down the dispersion before forming the homogeneous gel or paste, thereby extending the overall time required for the making process. Another drawback may include higher energy costs and extra time associated with heating. Yet another drawback may be that the current process requires the time-consuming and energy intensive heating/chilling in order to maximize the hydration of the thickening agents. Another traditional process for making oral care composition is by combining thickening agents and inorganic salts together into a vessel and mixing the resultant mixture while mixing for a cost prohibitive time or mixing in such a way to not fully utilize the added thickening agents. All of these options result in a less than optimized process or product.

Therefore, there is a need for an oral care composition making methodology that is capable of maximizing the hydration of the thickening agents while minimizing the processing time and/or energy costs associated with heating and/or cooling.

### SUMMARY OF THE INVENTION

In a first aspect, the present invention provides a process for making an oral care composition, comprising the steps of: a) hydrating a linear sulfated polysaccharide in water at a temperature not exceeding 50° C. to provide a hydrated homogeneous gel; and b) adding inorganic salts into the hydrated homogeneous gel. Optionally, the process further comprises the step c) of adding calcium-containing abrasive to the hydrated homogeneous gel, preferably the oral care composition comprises from 25% to 60%, preferably from 27% to 47%, of a calcium-containing abrasive by weight of the oral care composition, wherein preferably the calcium-containing abrasive is calcium carbonate.

In another aspect of the present invention, an oral care composition obtained by the above-described process is provided. It is desirable that the oral care composition does not develop phase separation, particularly after product storage.

In yet another aspect of the present invention, an oral care composition, preferably a dentifrice, obtained by the above-described process is provided having improved smoothness properties. It is desirable that the oral care composition comprises a linear sulfated polysaccharide, preferably a carrageenan, which has been hydrated in the absence of inorganic salts.

In yet another aspect of the present invention, use of an oral care composition obtained by the above-described process is provided for promoting or enhancing oral health.

While the specification concludes with claims that particularly point out and distinctly claim the invention, it is believed the present invention will be better understood from the following description.

### BRIEF DESCRIPTION OF THE DRAWINGS

While the specification concludes with claims particularly pointing out and distinctly claiming the invention, it is believed that the invention will be better understood from the following description of the accompanying FIGURES wherein:

FIG. 1 is a flow diagram for the process for making an oral care composition according to an embodiment of the present invention.

### DETAILED DESCRIPTION OF THE INVENTION

### Definitions

The term “oral care composition” as used herein means a product that in the ordinary course of usage is retained in the oral cavity for a time sufficient to contact some or all of the dental surfaces and/or oral tissues for purposes of oral activity. In one embodiment, the composition provides a benefit when used in the oral cavity. The oral care composition of the present invention may be in various forms including toothpaste, dentifrice, tooth gel, tooth powders, tablets, rinse, sub gingival gel, foam, mouse, chewing gum, lipstick, sponge, floss, prophy paste, petrolatum gel, or denture product. In one embodiment, the oral care composition is in the form of a paste or gel. In another embodiment, the oral care composition is in the form of a dentifrice. The oral care composition may also be incorporated onto strips or films for direct application or attachment to oral surfaces, or incorporated into floss.

The term “dentifrice” as used herein means paste, gel, powder, tablets, or liquid formulations, unless otherwise specified, that are used to clean the surfaces of the oral cavity. Preferably, the dentifrice compositions of the present invention are single phase compositions.

The term “mixing” as used herein refers to combining and further achieving a relatively greater degree of homogeneity thereafter.

The term “smoothness” as used herein refers to the uniform appearance devoid of lumps of an oral care composition, such as a dentifrice matrix, observed visually by un-aided eye.

The term “teeth” as used herein refers to natural teeth as well as artificial teeth or dental prosthesis.

The term “comprise”, “comprising” as used herein means that steps and ingredients other than those specifically mentioned can be added. This term encompasses the terms “consisting of” and “consisting essentially of”. The compositions of the present invention can comprise, consist of, and consist essentially of the essential elements and limitations of the invention described herein, as well as any of the additional or optional ingredients, components, steps, or limitations described herein.

As used herein, the articles including “a” and “an” when used in a claim, are understood to mean one or more of what is claimed or described.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, the term “substantially free of” refers to the presence of no more than 0.5%, preferably no more than 0.2%, and more preferably no more than 0.1%, of an indicated material in a composition, by total weight of such composition.

As used herein, the term “essentially free of” means that the indicated material is not deliberately added to the composition, or preferably not present at analytically detectable levels. It is meant to include compositions whereby the indicated material is present only as an impurity of one of the other materials deliberately added.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified. The term “weight percent” may be denoted as “wt %” herein. All molecular weights as used herein are weight average molecular weights expressed as grams/mole, unless otherwise specified. All temperatures are in Celsius degrees (° C.), unless specifically stated otherwise.

### Hydration of Carrageenan

The traditional making process for an oral care composition suffers from several disadvantages, in particular, lengthy production time, and/or significant energy consumption due to the heating and/or cooling. In today's environment of diminished resources, these conditions pose sustainability challenges and therefore a new manufacturing process is needed.

Accordingly, the making process of the present invention can significantly reduce the disadvantages described above. The present invention is directed to a process for making an oral care composition, especially a dentifrice containing linear sulfated polysaccharide which has been hydrated in the absence of inorganic salts. The process comprises the steps of: a) hydrating a linear sulfated polysaccharide in water at a temperature not exceeding 50° C., to provide a hydrated homogeneous gel; and b) adding inorganic salts to the hydrated homogenous gel.

The foregoing benefits are possible, in part, due to the reduction of the lengthy residence time from the heating and/or chilling typically needed in the traditional making process, which tends to increase the overall production time. The inventor have surprisingly discovered that adding the inorganic salts after the linear sulfated polysaccharide has been hydrated in water can maximize the hydration of linear sulfated polysaccharide, as well as speed up its hydration, even under the lower temperature conditions (i.e., ambient temperature) of the present invention. An advantage is to reduce the time and energy cost for the manufacturing process. By avoiding heating and/or cooling, it will be possible to save energy and cost for capital investment as well as save the time otherwise needed for heating/cooling down the solution. Another advantage is to provide an oral care product produced by this process that may exhibit a better smoothness on appearance based on the consumer's observation. Yet another advantage of the present invention is that by maximizing the hydration of the linear sulfated polysaccharide, it may result in a decrease in the levels of the raw material needed for the making process. This may also result in a more efficient binding of the linear sulfated polysaccharide to the other raw materials used in the process since the linear sulfated polysaccharide is fully hydrated under the lower temperature conditions of the present invention. Yet another advantage of the present invention is that, by maximizing the degree of hydration of the linear sulfated polysaccharide, it may also allow for the production of an oral care composition, preferably a dentifrice, having a high water content (e.g. >40 wt %) and a high carbonate content (e.g., >25 wt %).

Preferably, the linear sulfated polysaccharide present in the oral care composition is in the range from 0.01% to 15%, preferably 0.1% to 6%, more preferably from 0.1% to 3%, or most preferably from 0.5% to 2%, by weight of the oral care composition. A suitable example of a linear sulfated polysaccharide is carrageenan. Generally, carrageenan can vary based upon the degree of sulfation and may include: Kappa-carrageenan, Iota-carrageenan, and Lambda-carrageenan. Combinations of carrageenan can be used in the present invention. Preferably, the carrageenan is Iota-carrageenan.

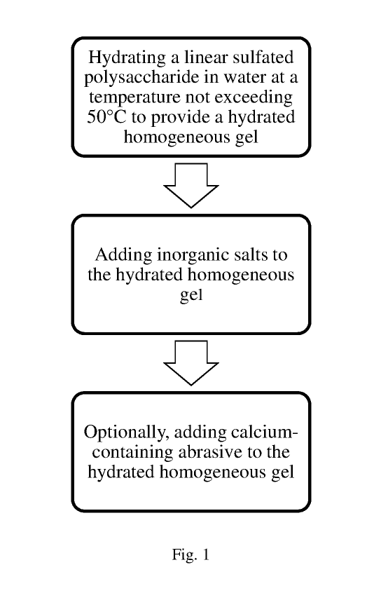

In one aspect, with reference to FIG. 1, the making process of the present invention can be a batch process. The hydration step a) can be conducted in a batch mixing vessel with a vacuum. Water is added into the batch mixing vessel and the carrageenan of desired amount is then added therein. Herein the water is pure water, without any intentionally added salts therein. Upon addition into water, the carrageenan is dispersed, swollen and hydrated under mechanical mixing. The carrageenan may be grounded or milled before addition into water. Alternatively, carrageenan can be used directly. The carrageenan may be added into the batch mixing vessel in portions such as for example, in three portions. Alternatively, the carrageenan may be added into the batch mixing vessel in pre-slurry form. The hydration can be conducted by using any mixing method used in the art, such as for example, agitation (e.g. mechanical agitation or gas-flow agitation) or shear mixing. In an embodiment, recirculation approach can be used to accelerate the dispersion and hydration.

The hydrating step a) is conducted at a temperature not exceeding 50° C., preferably at a temperature from 5° C. to 45° C., or more preferably from 15° C. to 40° C. In the most preferred embodiment, the hydration step a) can be conducted without heating, i.e. the hydration can be conducted at ambient temperature (where temperature ranging from 20° C. to 25° C.). Without using any separate heating device during the hydration step a), means that no subsequent cooling device is needed. As a result, the hydration step a) of the present invention has the benefit of saving time and cost for capital investment.

Preferably, the hydrating step a) is performed for a period of time from 1 minute to 2 hours, or more preferably from 2 minutes to 30 minutes. Preferably, the hydrating step is performed for a period time sufficient to maximize the hydration of carrageenan before step b) of the addition of inorganic salts.

### Inorganic Salts Addition

The making process of the present invention further comprises a step b) of adding inorganic salts into the hydrated homogeneous gel to make the oral care composition. Preferably, the inorganic salt used herein can be water-soluble metal salts only if they are orally acceptable. The inorganic salts can be added to the hydrated homogeneous gel in solid or powder form. Alternatively, the inorganic salts can be added to the hydrated homogeneous gel in a form of pre-dissolved aqueous solution.

The resource of inorganic salts can be from pH buffering agents, fluoride ion source, anti-caries agent, anti-microbial/anti-plaque agent, bleaching agent, anti-calculus agent, desensitizing agent, tooth substantive agent, chelating agent, surfactant, flavors, and combinations thereof. Preferably the inorganic salts can be selected from the group consisting of alkali metal salts, alkaline earth metal salts, ammonium salts and mixture thereof. In one embodiment, the inorganic salts used in the oral care composition comprise one or more selected from the group consisting of trisodium phosphate (TSP), monosodium phosphate (MSP), sodium monofluoride phosphate (MFP), tetrasodium pyrophosphate (TSPP), and combinations thereof. In some embodiments, the water-soluble metal salt is present at a level of from 0.01% to 10%, preferably from 0.2% to 5%, or more preferably from 0.5% to 2.5%, by weight of the oral care composition.

### Abrasive

The process of the present invention may further comprise a step c) of adding calcium-containing abrasive to the hydrated homogeneous gel to make the oral care composition. Preferably, the step c) of adding calcium-containing abrasive can be performed after step b). Preferably, the calcium-containing abrasive is selected from the group consisting of calcium carbonate, dicalcium phosphate, tricalcium phosphate, calcium orthophosphate, calcium metaphosphate, calcium polyphosphate, calcium oxyapatite, and combinations thereof. For example, the oral care composition may comprise from 25% to 60%, more preferably from 25% to 50%, even more preferably from 25% to 40%, yet even more preferably from 26% to 39%, alternatively from 27% to 47%, alternatively from 27% to 37%, alternatively from 30% to 35%, alternatively from 30% to 34%, alternatively combinations thereof, of a calcium-containing abrasive by weight of the composition.

In one embodiment, the calcium-containing abrasive is calcium carbonate. In a preferred embodiment, the calcium-containing abrasive is selected from the group consisting of fine ground natural chalk, ground calcium carbonate, precipitated calcium carbonate, and combinations thereof. In a more preferred embodiment, the calcium-containing abrasive is selected from fine ground natural chalk, ground calcium carbonate, and combinations thereof (at the aforementioned weight percentage ranges for calcium-containing abrasive).

Other abrasive that can be used in the oral care composition of the present invention may include fused silica abrasive, precipitated silica, rice hull silica, silica gels, aluminas, aluminium silicates, bentonite, water-insoluble phosphates including orthophosphates, polymetaphosphates, pyrophosphates, other inorganic particulates, and mixtures thereof.

### Thickening Agents

Besides Carrageenan, the oral care composition may further comprise other thickening agents selected from the group consisting of a carboxymethyl cellulose (“CMC”), a hydroxyethyl cellulose (HEC), a thickening silica, and combinations thereof. For instance, one commercially available form of CMC is CMC 2000S available from CPKelco. In another embodiment, the HEC has an average molecular weight range of 90,000 g/mol to 1,300,000 g/mol and an average degree of polymerization from 300 to 4,800. In another embodiment, the thickening silica is obtained from sodium silicate solution by destabilizing with acid as to yield very fine particles. One commercially available example is ZEODENT® branded silicas from Huber Engineered Materials (e.g., ZEODENT® 103, 124, 113 115, 163, 165, 167).

### Sweetener

The oral care compositions herein may comprise a sweetening agent. Suitable examples include sweeteners such as saccharin, dextrose, sucrose, lactose, maltose, levulose, aspartame, sodium cyclamate, D-tryptophan, dihydrochalcones, acesulfame, sucralose, neotame, and mixtures thereof. Sweetening agents are generally used in oral care compositions at levels of from 0.005% to 5%, by weight of the composition, alternatively 0.01% to 1%, alternatively from 0.1% to 0.5%, alternatively combinations thereof.

### Fluoride Ion Source

The oral care compositions herein may comprise an effective amount of an anti-caries agent. In one embodiment, the anti-caries agent is a fluoride ion source. The fluoride ion may be present in an amount sufficient to give a fluoride ion concentration in the composition at 25° C., and/or in one embodiment can be used at levels of from 0.0025% to 5% by weight of the composition, alternatively from 0.005% to 2% by weight of the composition, to provide anti-caries effectiveness. Representative fluoride ion sources include: stannous fluoride, sodium fluoride, potassium fluoride, amine fluoride, sodium monofluorophosphate, and zinc fluoride. In one embodiment, the oral care composition comprises a fluoride source selected from stannous fluoride, sodium fluoride, and mixtures thereof. In one embodiment, the fluoride ion source is sodium monofluorophosphate, and wherein the composition comprises 0.0025% to 2% of the sodium monofluorophosphate by weight of the composition, alternatively from 0.5% to 1.5%, alternatively from 0.6% to 1.7%, alternatively combinations thereof.

pH

Preferably, the oral care composition of the present invention has a pH of from 7.8 to 13, preferably from 8 to 12, alternatively greater than 8, alternatively greater than 9, alternatively from 9 to 11, alternatively from 9 to 10, or combinations thereof.

### pH Modifying Agent

The oral care compositions herein may comprise an effective amount of a pH modifying agent, alternatively wherein the pH modifying agent is a pH buffering agent. pH modifying agents, as used herein, refer to agents that can be used to adjust the pH of the oral care compositions to the above-identified pH range. pH modifying agents may include alkali metal hydroxides, ammonium hydroxide, organic ammonium compounds, carbonates, sesquicarbonates, borates, silicates, phosphates, imidazole, and mixtures thereof. Specific pH agents include monosodium phosphate (monobasic sodium phosphate or “MSP”), trisodium phosphate (sodium phosphate tribasic dodecahydrate or “TSP”), sodium benzoate, benzoic acid, sodium hydroxide, potassium hydroxide, alkali metal carbonate salts, sodium carbonate, imidazole, pyrophosphate salts, sodium gluconate, lactic acid, sodium lactate, citric acid, sodium citrate, phosphoric acid. In one embodiment, 0.01% to 3%, preferably from 0.1% to 1% of TSP by weight of the composition, and 0.001% to 2%, preferably from 0.01% to 0.3% of MSP by weight of the composition is used. Without wishing to be bound by theory, TSP and MSP may also have calcium ion chelating activity and therefore provide some monofluorophosphate stabilization (in those formulations containing monofluorophosphate).

### Anti-Calculus Agent

The oral care compositions may comprise an effective amount of an anti-calculus agent, which in one embodiment may be present from 0.05% to 50%, by weight of the composition, alternatively from 0.05% to 25%, alternatively from 0.1% to 15% by weight of the composition. One example is a pyrophosphate salt as a source of pyrophosphate ion. In one embodiment, the composition comprises tetrasodium pyrophosphate (TSPP) or disodium pyrophosphate or combinations thereof, preferably 0.01% to 2%, more preferably from 0.1% to 1% of the pyrophosphate salt by weight of the composition. Without wishing to be bound by theory, TSPP may provide not only calcium chelating thereby mitigating plaque formation, but also may also provide the additional benefit of monofluorophosphate stabilization (in those formulations containing monofluorophosphate).

### Surfactant

The oral care compositions herein may comprise a surfactant. The surfactant may be selected from anionic, nonionic, amphoteric, zwitterionic, cationic surfactants, or mixtures thereof. The composition may comprise a surfactant at a level of from 0.01% to 10%, preferably from 0.05% to 9%, or more preferably from 0.1% to 5% by weight of the total composition. Non-limiting examples of anionic surfactants may include those described at US 2012/0082630 A1 at paragraphs 32, 33, 34, and 35. Non-limiting examples of zwitterionic or amphoteric surfactants may include those described at US 2012/0082630 A1 at paragraph 36; cationic surfactants may include those described at US 2012/0082630 A1 at paragraph 37; and nonionic surfactants may include those described at US 2012/0082630 A1 at paragraph 38. Preferred surfactant is sodium lauryl sulfate (SLS).

### Less or No Humectants

The oral care composition herein may be substantially free or essentially free of humectants, or alternatively contain low levels of humectants. The term “humectant”, for the purposes of the present invention, includes edible polyhydric alcohols such as glycerin, sorbitol, xylitol, butylene glycol, propylene glycol, and combinations thereof. In one example, the oral care composition is substantially free of either sorbitol or glycerin or both. In yet another example, the oral care composition is substantially free of any humectant selected from the group consisting of glycerin, sorbitol, xylitol, butylene glycol, propylene glycol, and combinations thereof. Alternatively, the oral care composition comprises from 0% to less than 5% of humectants by weight of the composition, preferably from 0% to 3%, alternatively from 0% to 2%, alternatively from 0% to 1%, alternatively less than 10%, or less than 9%, 8%, 7%, 6%, 4%, 3%, 2%, 1%, or less than 0.5%; or greater than 0.1%, or greater than 0.2%, 0.5%, 1%, or 1.5%; or combinations thereof, by weight of the composition.

### Colorant

The oral care composition herein may comprise a colorant. Titanium dioxide is one example of a colorant. Titanium dioxide is a white powder which adds opacity to the composition. Optionally, titanium dioxide may comprise from 0.25% to 5%, by weight of the composition.

### Flavorant

The oral care composition herein may comprise from 0.001% to 5%, alternatively from 0.01% to 4%, alternatively from 0.1% to 3%, alternatively from 0.5% to 2%, alternatively 1% to 1.5%, alternatively 0.5% to 1%, alternatively combinations thereof, of a flavorant composition by weight of the composition. The term flavorant composition is used in the broadest sense to include flavor ingredients, or sensates, or sensate agents, or combinations thereof. Flavor ingredients may include those described in US 2012/0082630 A1 at paragraph 39; and sensates and sensate ingredients may include those described at paragraphs 40-45. Excluded from the definition of flavorant composition is “sweetener” (as described above).

### Water

Water is commonly used as a carrier material in an oral care composition due to its many benefits. For example, water is useful as a processing aid, is benign to the oral cavity and assists in quick foaming of toothpastes.

Water may be added as an ingredient in its own right or it may be present as a carrier in other common raw materials such as, for example, sorbitol and sodium lauryl sulfate. The term “total water content” as used herein means the total amount of water present in the oral care composition, whether added separately or as a solvent or carrier for other raw materials but excluding that which may be present as water of crystallization in certain inorganic salts.

The oral care composition of the present invention comprises at least 20% of a total water content. In an embodiment, the oral care composition comprises from 20% to 75% of a total water content. In another embodiment, the oral care composition comprises from 40% to 60% of a total water content. In other embodiments, the compositions include from 45% to 65%, alternatively from 40% to 60%, alternatively from 50% to 70%, alternatively from 50% to 60%, alternatively from 45% to 55%, alternatively from 55% to 65%, alternatively from 55% to 60%, alternatively 55%, alternatively combinations thereof, of a total water content. Preferably, the water is USP water.

The present oral care composition can comprise the usual and traditional ancillary components that are known to one skilled in the art. It will be appreciated that selected components for the oral care compositions must be chemically and physically compatible with one another.

### Oral Care Composition

Non-limiting examples of oral care composition resulting from the making process according to the present invention are selected from the group consisting of: toothpaste, dentifrice, tooth gel, tooth powders, tablets, rinse, sub gingival gel, foam, mouse, chewing gum, lipstick, sponge, floss, prophy paste, petrolatum gel, and denture product, preferably dentifrice.

Phase stability means visually (i.e., to the unaided eye) having no liquid separated from the oral care composition (e.g., toothpaste) body over a defined period of time under ambient conditions. Preferably, the oral care composition of the present invention does not exhibit phase separation, particularly after storage. Preferably, the oral care composition of the present invention does not exhibit phase separation after 1 month at 40° C.; preferably after 2 months at 40° C.; or more preferably after 6 months at 40° C.

### Method of Use

The present invention also relates to methods for treating the oral cavity comprising the step of administering to the oral care cavity an oral care composition according to the present invention. The present invention also relates to a method of treating tooth enamel comprising the step of contacting teeth with the oral care composition according to the present invention. In an embodiment, the term “treating” refers to cleaning and polishing teeth. The method of use herein comprises contacting a subject's dental enamel surfaces and oral mucosa with the oral care compositions according to the present invention. The method of treatment may be by brushing with a dentifrice or rinsing with a dentifrice slurry or mouth rinse. Other methods include contacting the topical oral gel, mouthspray, toothpaste, dentifrice, tooth gel, tooth powders, tablets, subgingival gel, foam, mouse, chewing gum, lipstick, sponge, floss, petrolatum gel, or denture product or other form with the subject's teeth and oral mucosa. Depending on the embodiment, the oral care composition may be used as frequently as toothpaste, or may be used less often, for example, weekly, or used by a professional in the form of a prophy paste or other intensive treatment. The present invention is also directed to the use of the oral care composition of the present invention for promoting or enhancing oral health.

### Test Methods

### Test Method 1: Viscosity Test

The viscosity as a function of different shear rates of the examples is measured using a RheolabQC Rheometer MC1 with Z3 couette geometry (Anton Paar, Graz, Austria). The results are reported in units of Pa·s.

### Testing Procedure:

1. Set temperature at 20° C.
2. Set up software RheoSolve (V2.11 Copyright© 1997 by Anton PAAR Austria software.), using MS Z3 DIN system.
3. Load the sample into the rheometer.
4. Run the test and read data.
5. Remove the sample.
   Parameter setting:
   CSR Speed Ramp n1=0.39 RPM, n2=500 RPM, measurement time 2.0 secs, temperature 20° C., equilibrium time 100 secs.

### Test Method 2: Phase Stability Test

The phase stability of the oral care composition is evaluated by placing the composition in a tube with a sealed cap for up to at least 1 month, preferably for at least 2 months, more preferably for at least 6 months under atmospheric pressure at 40° C. The assessment is conducted by a naked or unaided eye.

### EXAMPLES

The following examples describe and demonstrate embodiments within the scope of the invention. The examples are given solely for the purpose of illustration and are not to be construed as limitations of the present invention, as many variations thereof are possible without departing from the spirit and scope of the invention.

### Example 1: Comparison Tests Showing Impact of Different Salt Addition Order on Viscosity

Example A is a non-limiting example of the making process according to the present invention. Examples B to D are comparative examples according to traditional process, which are outside of the scope of the present invention.

### Example A

346.92 mL deionized water is added into a 500 mL vessel and 8 g of carrageenan (Viscarin© 1280 from FMC Biopolymer Co, Philadelphia, US) is added therein at ambient temperature (around 25° C.). The mixture is stirred with a stirrer (IKA® EUROSTAR POWER control-visc) at 400 RPM for 15 minutes and a hydrated homogeneous gel is formed. Pre-dissolved 9.08 g of sodium chloride salt (0.9%) in 36 mL deionized water is then added to the hydrated homogeneous gel and stirred for 2 minutes. The viscosity of the resultant mixture is measured.

### Example B

1 g sodium chloride salt (0.1 wt %) is added in 391 mL deionized water contained in a 500 mL vessel (or beaker) at ambient temperature (around 25° C.). 8 g of carrageenan (Viscarin© 1280 from FMC Biopolymer Co) is then added therein. The mixture is stirred with a stirrer at 400 RPM for 15 minutes and a gel mixture is formed. The viscosity of the resultant gel mixture is measured.

### Example C

Example C is made in the same manner as Example B, except that 9.08 g sodium chloride salt (0.9 wt %) is added in 382.92 mL deionized water. The viscosity of the resultant gel mixture is measured.

### Example D

Example D is made in the same manner as Example C, except that the mixture is stirred with a stirrer at 400 RPM and heated to 60° C. then remaining for 15 minutes. The mixture then allowed to cool down to ambient temperature and a gel mixture is formed. The viscosity of the resultant gel mixture is measured.

The viscosities of all Examples A-D measured using the viscosity test method described herein at a temperature of 20° C. are listed in Table 1 below.

TABLE 1
Viscosity Measurements

| | Carrageenan | Sodium | Sodium salts | Hydration | Viscosity at |
|---|---|---|---|---|---|
| | content | salts | addition (before or | Temperature | shear rate of |
| Example | (wt %) | (wt %) | after hydration) | (° C.) | 0.5 s⁻¹ (Pa · s) |
| A (Inventive) | 2 | 0.9 | after | Ambient | 81.19 |
| B (comparative) | 2 | 0.1 | Before | Ambient | 9.65 |
| C (comparative) | 2 | 0.9 | before | Ambient | —* |
| D (comparative) | 2 | 0.9 | before | 60 | 137.04 |

*too low to be detected using viscosity test method described herein.

The result of Inventive Example A shows that addition of sodium salt after the hydration of carrageenan achieves a desired viscosity (81.19 Pa·s). However, by comparison of Examples A and B, if the sodium salt is added before the hydration of carrageenan, even if present in a small amount (0.1 wt %), the viscosity of carrageenan dispersion decreases significantly (from 81.19 Pa·s to 9.65 Pa·s). Furthermore, when more sodium salt is present (e.g., 0.9 wt % for Example C), the viscosity is too small to be detected using the viscosity test method described herein.

Example D demonstrates a traditional process for making oral care composition containing carrageenan, in which sodium salt is added before carrageenan addition and the hydration is conducted at a temperature of 60° C. If comparing Example C, D, and A, it can be seen that the inventive process of delaying the addition of sodium salt (Example A) can enable the hydration of carrageenan even in ambient temperature without the need for heating while achieving a comparative viscosity versus traditional process. In contrast, a heating step (to at least 60° C.) is required for a traditional process (Example D), otherwise the hydration is inhibited and viscosity of carrageenan mixture is too low to be detected (Example C).

### Example 2: Illustrative Example

A dentifrice composition according to the present invention is prepared by the following process. The formulation is shown in Table 2.
1) Mix MFP, MSP, TSP, TSPP into USP water in a separate container until clear pre-mixed solution is achieved.
2) To a batch mixing vessel, add USP water and flavor, and then Carrageenan and CMC are added therein at ambient temperature (around 25° C.). The mixture is mixed and homogenized with high shear mixer/homogenizer under vacuum. The resulted mixture is further mixed for 10 minutes.
3) The pre-mixed solution of step 1) is then added to the mixture of step 2), and is mixed for further 5 minutes.
4) Then thickening silica is added into the vessel and the mixture is further mixed for 5 minutes.
5) Then calcium carbonate is added into the vessel and mixed for at least 10 minutes under vacuum.
6) Then add sodium lauryl sulfate (SLS) and remaining flavor to the vessel. Deaerate the batch mixing vessel for at least 10 minutes under vacuum. The product is packed into a tube.

TABLE 2
Dentifrice Composition

| Ingredients | Dentifrice Composition (wt %) |
|---|---|
| Tetrasodium Pyrophosphate | 0.6 |
| Carrageenan | 1.2 |
| Sodium Carboxymethyl Cellulose | 0.9 |
| Thickening Silica | 2.6 |
| Preservatives | 0.1 |
| Sodium Saccharine | 0.2 |
| Sodium Monofluorophosphate | 1.1 |
| Sodium Monophosphate | 0.1 |
| Sodium Triphosphate | 0.4 |
| Calcium Carbonate | 32 |
| Sodium Lauryl Sulfate | 4 |
| Flavor | 0.8 |
| Deionized Water | 56 |
| Total: | 100 |

The phase stability test shows that the dentifrice composition exhibits no phase separation after being stored for 6 months at 40° C.

The dentifrice composition of the present invention is expected to have improved smoothness compared to a comparative composition made by the traditional method wherein hydration of the carrageenan is conducted in the presence of inorganic salts.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for making an oral care composition, comprising the steps of:
a) hydrating a linear sulfated polysaccharide in water at a temperature not exceeding 50° C. to provide a hydrated homogeneous gel; and
b) adding inorganic salts to the hydrated homogeneous gel.

2. The process according to claim 1, wherein the hydrating step a) is conducted at a temperature from 5° C. to 45° C., preferably from 15° C. to 40° C.

3. The process according to claim 1, wherein the hydrating step a) is performed with a residence time of from 1 minute to 2 hours.

4. The process according to claim 1, wherein the linear sulfated polysaccharide is a carrageenan.

5. The process according to claim 4, wherein the carrageenan is selected from the group consisting of Kappa-carrageenan, Iota-carrageenan, Lambda-carrageenan and combinations thereof.

6. The process according to claim 1, wherein the oral care composition does not exhibit phase separation after 1 month at 40° C.

7. The process according to claim 1, wherein the inorganic salts are water-soluble metal salts, selected from the group consisting of alkali metal salts, alkaline earth metal salts, ammonium salts and mixture thereof, or more preferably selected from the group consisting of trisodium phosphate, monosodium phosphate, sodium monofluoride phosphate, tetrasodium pyrophosphate, and mixtures thereof; wherein the water-soluble metal salt is present at a level of from 0.01% to 10%, by weight of the oral care composition.

8. The process according to claim 1, further comprising the step c) of adding calcium-containing abrasive to the hydrated homogeneous gel.

9. The process according to claim 1, wherein the oral care composition comprises from 20% to 75% of a total water content by weight of the oral care composition.

10. The process according to claim 1, wherein the oral care composition comprises 0 to 5% of humectant by weight of the composition.

11. The process according to claim 1, wherein the oral care composition is a dentifrice.

12. A dentifrice produced by the process according to claim 1.

13. A method of treating tooth enamel comprising the step of contacting teeth with the dentifrice according to claim 12.

14. Use of the dentifrice according to claim 12 in promoting or enhancing oral health.
